(19) United States
(12) Patent Application Publication
(10) Pub. No.: US 2005/0177902 A1
(43) Pub. Date: Aug. 11, 2005

(54) Methods of producing specialty grain to segment markets

(76) Inventors: Troy T. Lohrmann, Lake Villa, IL (US); Jean C. Kridl, Davis, CA (US); Thomas J. Savage, Sacramento, CA (US); Daniel J. Dyer, Kildeer, IL (US)

Correspondence Address: RENESSEN LLC;Suite 220, 520 Lake Cook Road, Deerfield, IL, 60015, US

(21) Appl. No.: 10/991,889

(22) Filed: Nov. 18, 2004

Related U.S. Application Data
(60) Provisional application No. 60/520,912, filed on Nov. 18, 2003.

(51) Int. Cl. A01H 1/00; C12N 15/82; A01H 5/00; G06F 17/60

(52) U.S. Cl. 800/281; 800/320.1; 705/1

(57) ABSTRACT

The present invention provides a method of segmenting a market for a grain by manipulating one or more phenotypic characteristics of the grain. The phenotypic characteristics include oil content, pigmentation potential, iodine value, and linoleic acid content.

The present invention relates to the general fields of genetics, grain composition, and specifically to the production of novel corn for use in segmenting grain markets.

Certain nutritional traits in grains have a different commercial value based on the animal species the grain is fed to. For example, many broiler producers feed energy dense diets, consisting of supplemental fat, which compensates for the lower amount of digestible (metabolizable) energy in soybean meal as compared to corn. Poultry rations generally contain a higher proportion of soybean meal, as compared to growing swine, to supplement that animal's higher amino acid requirement that can not be met by corn alone. Swine have a longer gastro-intestinal tract compared to poultry that allows for more complete digestion of fibrous ingredients such as soybean meal. Pork producers, therefore, do not necessarily need to add supplemental fat to diets since the metabolizable energy of soybean meal is similar to that of corn. Although a grain trait such as high oil would benefit the production of both, it would have a higher value for poultry as compared to swine, as it would eliminate part or all the need to supplement fat in poultry rations.

Many large livestock operations have multiple species under production, such as a large producer of both broiler meat and pork. Additionally, a feed mill servicing that producer often manufactures feed for both pigs and chickens. Therefore, a feed ingredient containing a particular trait (such as high oil corn) may be valued and negotiated for purchase at a lesser price based on the species that would get the least value from the trait. Thus, maintaining the total or true market value of a value added feed ingredient, which would be fed to both species, is improbable. For example, with a higher oil corn (e.g., greater than or equal to 5.0% oil on a dry matter basis) having less value to swine than poultry, the situation exists, where a producer could negotiate the lower trait price based on a bias for swine. However, all high oil corn purchases could then be fed to poultry, as monitoring the use of the grain would be difficult by the supplier of high oil corn. Hence, the true market value for that high oil corn as a poultry feed ingredient would be lost to the supplier.

Recent advances in plant breeding and biotechnology have enabled the agribusiness industry to supply corn grain with improved nutritional traits as a feed ingredient to the markets outlined above. However, the agribusiness industry is understandably reticent to make the substantial investments necessary to create new products in view of the readily interchangeable nature of diets between higher and lower value food animals. Accordingly, there exists a need to have a method to distinguish feed designated for different food animals so that feed providers can capture the true market value of these improved grains.

### SUMMARY OF THE INVENTION

The present invention includes and provides a method of segmenting a market for a grain, comprising manipulating at least one phenotypic characteristic of the grain. The manipulated grain is more adapted to a first market segment and less adapted to a second market segment, as compared to a non-manipulated grain. The preferred phenotypic characteristics of the present invention include but are not limited to oil content, linoleic acid content, pigmentation potential, and iodine value.

The present invention further includes a method of segmenting a market for a grain comprising manipulating at least one phenotypic characteristic of the grain, wherein the manipulated grain is more adapted to a first market segment and less adapted to a second market segment, as compared to a non-manipulated grain, wherein at least one phenotypic characteristic is undesirable to said second market segment. In one embodiment, the first market segment is swine and the second market segment is poultry. Examples of preferred manipulated grains that are more adapted to a swine market than to a poultry market include, but are not limited to (a) grain having an oil content increased to between about 6% and about 30%, and a pigmentation potential less than that of standard yellow corn; (b) grain having a pigmentation potential less than that of standard yellow corn; (c) grain having an iodine value less than about 70, and a pigmentation potential less than that of standard yellow corn; (d) grain having an oil content increased to between about 6% and about 30%, and an iodine value less than about 70; and (e) grain having an oil content increased to between about 6% and about 30%, a pigmentation potential less than that of standard yellow corn, and an iodine value less than about 70.

The present invention further includes a method of segmenting a market for a grain comprising manipulating at least one phenotypic characteristic of the grain, wherein the manipulated grain is preferably more adapted to a first market segment and less adapted to a second market segment, as compared to a non-manipulated grain, wherein at least one phenotypic characteristic is undesirable to said second market segment, and wherein the first market segment is poultry and the second market segment is swine. Examples of manipulated grains more adapted to a poultry market than to a swine market include, but are not limited to, (a) grains having a pigmentation potential greater than that of standard yellow corn; and (b) grains having an oil content increased to between about 6% and about 30%, and a pigmentation potential of greater than that of standard yellow corn.

A further embodiment of the present invention is a method of segmenting a market for a grain meal prepared from grain in which at least one phenotypic characteristic of the grain has been manipulated and the grain meal is more adapted to a first market segment and less adapted to a second market segment as compared to a grain meal prepared from non-manipulated grain, and wherein at least one phenotypic characteristic is undesirable to said second market segment.

The present invention further includes improved methods of producing a grain meal, wherein the method comprises manipulating a corn plant to have at least one phenotypic characteristic altered and wherein the altered phenotypic characteristic makes the meal more adapted to a first market segment and less adapted to a second market segment and wherein said phenotypic characteristic is undesirable to said second market segment.

### Definitions

The following definitions are helpful in understanding the specification and claims. The definitions provided herein should be borne in mind when these terms are used in the following examples and throughout the instant application. β-CAROTENE CONTENT means the concentration of the provitamin form of vitamin A in grain.

CORN MEAL means the direct product of grinding or crushing corn grain and without any supplementation.

DRY MATTER BASIS is the concentration (e.g., expressed as percent (w/w), mg/kg or ppm) of a nutrient or compound in grain, meal, food, or feed on a zero percent moisture basis.

EMASCULATE means the removal of plant male sex organs or the inactivation of the organs with a chemical agent or a cytoplasmic or nuclear genetic factor conferring male sterility.

GRAIN comprises mature corn kernels produced by commercial growers for purposes other than propagating the species.

GRAIN MEAL means the direct product of grinding or crushing grain and without any supplementation.

HYBRID refers to the progeny of a cross fertilization between parents belonging to different genotypes, or the first generation offspring of a cross between two homozygous individuals differing in one or more genes.

INBRED refers to a pure line usually originating by self-pollination and selection.

IODINE VALUE is a means to chemically measure the degree of unsaturation (C—C double bonds) of a fat or lipid. Iodine value is a measure of the unsaturation of fats and oils and is expressed in terms of the number of centigrams of iodine absorbed per gram of sample. Iodine value is calculated as equal to (% 16:1 hexadecanoic acid×0.95)+(% 18:1 octadecenoic acid×0.86)+(% 18:2 octadecadienoic acid×1.732)+(% 18:3 octadecatrienoic acid×2.616)+(% 20:1 eicosenoic acid×0.785)+(% 22:1 docosenoic acid×0.723).

KERNEL is the corn caryopsis comprising a mature embryo and endosperm, which are products of double fertilization.

LINOLEIC ACID CONTENT is the amount of an eighteen (18) carbon, two C—C double bond (polyunsaturated) fatty acid in the grain, grain meal, or oil, expressed as a percent on a dry matter basis.

METABOLIZABLE ENERGY refers to a system of describing the available energy content of food and the requirement of an animal. Metabolizable energy is the gross energy of a feed minus the energy lost in the excreta. Gross energy is defined as the energy liberated when a feed is burnt in oxygen.

OIL CONTENT (%) is the amount of the kernel that is oil, expressed as a percentage on a dry matter basis.

PALMITATE CONTENT is the amount of a sixteen (16) carbon, no C—C double bond (saturated) fatty acid in the grain, grain meal, or oil expressed as a percentage on a dry matter basis.

PIGMENT CONTENT is the concentration of a component of the diet that can impart a skin, lipid, shell, or meat color characteristic when included in the diet of various animal and fish specie. The pigment content of corn includes all pigments contained within the kernel, including but not limited to xanthophylls, lutein, carotenoids, anthrocyanins, and astaxanthin, and is represented in parts per million (ppm) or mg/kg. For example, standard yellow corn has a pigment content consisting of approximately 20 ppm xanthophylls.

PIGMENTATION POTENTIAL is the perceived or measured effect of a feed component, when consumed by an animal or fish, to impart a change in the color of that animal's or fish's skin, lipid layer, shell, or meat, wherein standard yellow corn serves as the reference component. Any animal feed component that has an equivalent ability to impart a pigmentation of the animal or fish skin, lipid layer, shell, or meat, equal to that of an equal weight of standard yellow corn, would have a pigmentation potential equal to that of standard yellow corn. Any feed component that imparts a pigmentation greater than that of an equal weight of standard yellow corn would have a pigmentation potential greater than that of standard yellow corn. Conversely, any animal feed component that imparts a pigmentation less than that of an equal weight of standard yellow corn would have a pigmentation potential less than that of standard yellow corn.

PROTEIN (%) is the amount of the kernel that is crude protein, expressed as a percentage on a dry matter basis.

SEED refers to mature corn kernels produced for the purpose of propagating the species.

STANDARD YELLOW CORN is defined as yellow corn containing not more than 5% corn of other colors. Yellow kernels of corn with a slight tinge of red are considered standard yellow corn. Unless otherwise noted, in the context of this invention, standard yellow corn shall have the same definition as specified by the United States Grain Standards Act.

STEARATE CONTENT is the amount of an eighteen (18) carbon, no C—C double bond (saturated) fatty acid in the grain, grain meal, or oil expressed as a percentage on a dry matter basis.

XANTHOPHYLL CONCENTRATION is the amount of xanthophyll in parts per million (ppm).

As used herein the phrase “segmenting a market” is to be understood as the division of an overall market for a grain into groups with common characteristics. For example, in the livestock industry, a common grain used for feeding broilers and swine could be segmented in to different groups based upon the nutritional requirements of the animal species. An example would be the different nutritional requirement for linoleic acid, where the NRC requirement is significantly higher for poultry than swine. The linoleic acid content of the grain would thus have more value in a poultry feed as compared to a swine feed. Thus the market for a grain for swine and poultry feed could be segmented based on the content of linoleic acid.

Unless otherwise defined, all technical and scientific terms used herein have the same meaning as commonly understood by one of ordinary skill in the art to which this invention belongs. Although methods and materials similar or equivalent to those described herein can be used to practice the present invention, suitable methods and materials are described below. All publications, patent application, patents, and other references mentioned herein are incorporated by reference in their entirety. In case of conflict, the present specification, including definitions, will control. In addition, the materials, methods, and examples are illustrative only and not intended to be limiting.

### DETAILED DESCRIPTION OF THE PREFERRED EMBODIMENTS

It is to be understood that both the foregoing summary and the following detailed description are exemplary and explanatory only and are not restrictive of the present invention as claimed.

The present invention provides methods of segmenting a market for a grain, such as corn. The method of the present invention comprises selecting or modifying one or more phenotypic characteristics of the grain which results in the grain and or grain meal being more adapted to one market segment, and less adapted to a second market segment. The concept of grain meal being more or less adapted to one market or another is a function of the relative nutritive, and therefore economic, impacts of a feed on the food animals that are the focus of the markets, and will be further explained herein.

Phenotypic Characteristics

As used herein, the phrase “high oil corn” refers to corn grain comprising greater than about 6 wt. %, preferably at least about 7 wt. %, and preferably at least about 8 wt. % oil, on a dry matter basis. A high oil corn has an elevated level of oil as compared to conventional yellow #2 corn, which has an oil content of about 3 wt. % to about 5 wt. %. Additionally, the total oil content of corn grain suitable for the invention can be, for example, grain having an oil content preferably at least about 9 wt. %, preferably at least about 11 wt. %, preferably at least about 12 wt. %, preferably at least about 15 wt. %, preferably at least about 18 wt. %, preferably at least about 20 wt. % oil, preferably from about 8 wt. % to about 20 wt. % oil, preferably from about 10 wt. % to about 20 wt. % oil, or preferably from about 14 wt. % to about 30 wt. % oil, and values within those ranges.

Corn grain having an elevated total oil content is identified by any of a number of methods known to those of skill in the art. The oil content of grain can be determined using American Oil and Chemical Society Official Method, 5^{th} edition, March 1998 (referred to herein as AOCS). AOCS method Ba3-38 quantifies substances that are extracted by petroleum ether under conditions of the test. The oil content or concentration is the weight percentage of the oil with respect to the total weight of the seed sample. Oil content may be normalized and reported at any desired moisture basis.

Other suitable methods for identifying high oil corn grain are described herein. According to one method, corn ears are selected using a near infrared (NIR) detection to select corn ears having corn kernels with elevated oil levels. Likewise, NIR detection can also be used to select individual corn kernels having elevated levels of oil. However, selecting individual ears and/or kernels having elevated oil content may not be cost effective in identifying high oil kernels suitable for processing using methods described herein. Generally, corn seed producing corn plants that yield grain having elevated total oil concentrations is planted and harvested using known farming methods. Methods for developing corn inbreds, hybrids, transgenic species, and populations that generate corn plants producing grain having elevated oil concentrations are known and described in Lambert (Specialty Corn, CRC Press Inc., Boca Raton, Fla., pp. 123-145 (1994)).

As used herein, the phrase “low pigment corn” refers to corn having a pigmentation potential less than that of standard yellow corn.

Preferred examples of low pigment corn of the present invention are white corn hybrids, including those of 1851W and E8272 (Wilson Genetics, Harland, Iowa), as described in U.S. Patent Application 20030066106. Additionally, the hybrid corn plants produced from the hybrid corn seeds, and variants, mutants and modifications of 1851 W and/or E8272, and similarly classified and characterized hybrids, are also preferred examples of low pigment corn of the present invention. This present invention may also relate to the use of such hybrids in producing other hybrids, e.g., three-way or double cross hybrids. The terms variant, trivial modification, and mutant refer to a hybrid seed where a plant produced by that hybrid seed is phenotypically similar to, for example, the 1851W and E8272 hybrids.

The inbred parents of the illustrative hybrid 1851 W include a tropical, and preferably, male parent WICY226C, and a domestic, and preferably, female parent WEBF428C. The inbred parents of the illustrative hybrid E8272 include a tropical, and preferably, male parent WICY418C, and a domestic, and preferably, female parent WEBF428C. The tropical inbred parents are selected for grain characteristics such as hardness, disease resistance, and diversity, but show good yield when crossed with U.S. derived stiff stalks. As used herein, the term “tropical” refers to germplasm originally collected from regions outside of the United States. The hybrids are selected from various inbred crosses based upon one or more grain characteristics, for example bright white kernel color.

As used herein “high pigment corn” refers to corn having a pigmentation potential greater than that of standard yellow corn. In one embodiment of the present invention, high pigment corn may be produced by increasing the concentration of carotenoid compounds, either endogenous or non-native compounds introduced through metabolic engineering technology. Carotenoids are pigments, yellow-orange-red lipids, with a variety of biological applications. Carotenoid hydrocarbons are referred to as carotenes, whereas oxygenated derivatives are referred to as xanthophylls. The carotenoid pathway in plants produces carotenes, such as α- and β-carotene, and lycopene, as well as xanthophylls, such as lutein.

The pathway for biosynthesis of the carotenoids has been studied in higher plants and the biosynthetic pathway has been elucidated. For examples, see, Britton, Biosynthesis of carotenoids, pp. 133-182, In T. W. Goodwin (ed.), Plant pigments, (1988). Academic Press, Inc. (London), Ltd., London. Carotenoid biosynthesis genes have also been cloned from a variety of organisms including Erwinia uredovora (Misawa et al., J. Bacteriol., 172: 6704-6712 (1990)); Erwinia herbicola (PCT Application WO 91/13078; Armstrong et al., Proc. Natl. Acad. Sci. (U.S.A.), 87: 9975-9979 (1990)); R. capsulatus (Armstrong et al., Mol. Gen. Genet., 216: 254-268 (1989), Romer et al., Biochem. Biophys. Res. Commun., 196: 1414-1421 (1993)); Thermus thermophilus (Hoshino et al., Appl. Environ. Microbiol., 59: 3150-3153 (1993)); the cyanobacterium Synechococcus sp. (Genbank accession number X63873), and PCT Application WO 96/13149 and the references cited therein.

Carotenoid biosynthetic genes from a variety of sources can be transformed into corn plants to increase the carotenoid concentration in the plant. U.S. Patent Application 20020092039 demonstrates that transformation of a plant with an early carotenoid biosynthesis gene, phytoene synthase, leads to a significant increase in the flux through the carotenoid pathway, resulting in an increased carotenoid concentration in the seeds. High carotenogenic corn plants generated in this manner can be crossed with high oil corn plants and plants containing both the high oil and high carotenoid phenotype can be selected.

Carotenoid compounds in corn can be detected by analytical methodologies well known in the art. For example, methods using high performance liquid chromatography (HPLC, Weber, J. Am. Oil Chem. Soc., 64(8): 1129-1134 (1987)) and spectrophotometric determination (White et al., Ind. Eng. Chem., Anal. Ed., 14: 798-801 (1942)).

The iodine value of corn grain measures the degree of unsaturation of the lipid fraction of the seed. Therefore, corn grain having a greater proportion of the lipid fraction as 18:0 (stearic acid) or 18:1 (oleic acid) and correspondingly less 18:2 (linoleic acid) and 18:3 (linolenic), as compared to that of standard yellow corn, would have a lowered iodine value. Methods that increase the proportion of stearic and/or oleic to linolenic would thus lower the iodine value of the corn grain.

Numerous methods of altering the ratio of saturated and unsaturated fatty acids in the lipid fraction of plants have been published. Higher plants appear to synthesize fatty acids via a common metabolic pathway. In developing corn seeds, where fatty acids are attached to glycerol backbones, the fatty acid synthetase (FAS) pathway is located in the proplastids. The first step in initiation stage of fatty acid synthesis is the carboxylation of the 2 carbon acetyl-CoA to form the 3-carbon β-ketoacid malonyl-CoA by acetyl-CoA carboxylase (ACCase). The ACCase step is irreversible, so once this step is accomplished, the resultant carbon compound is committed to fatty acid synthesis. All subsequent steps are catalyzed by the FAS. Malonyl-ACP is synthesized from malonyl-CoA and acylcarrier protein (ACP) by the enzyme malonyl-CoA:ACP transacylase. An acetyl moiety from acetyl-CoA is joined to a malonyl-ACP in a condensation reaction catalyzed by β-ketoacyl-ACP synthase III. Elongation of acetyl-ACP to 16- and 18-carbon fatty acids involves the cyclical action of the following sequence of reactions. After acetyl-CoA is condensed with malonyl-ACP using β-ketoacyl-ACP synthase, a β-ketoacyl-ACP is formed. The keto group on the β-ketoacyl-ACP is then reduced to an alcohol by β-ketoacyl-ACP reductase. The alcohol is removed in a dehydration reaction to form an enoyl-ACP by β-hydroxyacyl-ACP dehydratase. Finally, the enoyl-ACP is reduced to form the elongated saturated acyl-ACP by enoyl-ACP reductase.

The enzyme β-ketoacyl-ACP synthase I catalyzes elongation up to palmitoyl-ACP (C16:0), which is generally the end product from which other types of fatty acids are made. The enzyme β-ketoacyl-ACP synthase II catalyzes the final elongation of palmitoyl-ACP to stearoyl-ACP (C18:0).

Common plant unsaturated fatty acids, such as oleic, linoleic and α-linolenic acids, originate from the desaturation of stearoyl-ACP to form oleoyl-ACP (C18:1) in a reaction catalyzed by a soluble plastid enzyme, Δ-9 desaturase (also often referred to as “stearoyl-ACP desaturase”). Molecular oxygen is required for desaturation and reduced ferredoxin serves as an electron co-donor.

Additional desaturation is effected sequentially by the actions of membrane bound Δ²-desaturase and Δ¹⁵-desaturase. These desaturases thus create mono- or polyunsaturated fatty acids, respectively.

The identification of enzyme targets and useful plant sources for nucleic acid sequences of such enzyme targets capable of modifying fatty acid compositions are the subject of recent patents. For example, U.S. Pat. No. 6,350,934 discloses the use of ribozymes to down-regulate the Δ⁹-desaturase gene, and U.S. Pat. No. 6,566,584 discloses the overexpression of acetyl-CoA synthetase. Ideally, an enzyme target will be amenable to one or more applications alone or in combination with other nucleic acid sequences, relating to the ratio of saturated to unsaturated fatty acids in the fatty acid pool, and/or to novel oil compositions as a result of the modifications to the fatty acid pool.

Additionally, non-transgenic high oleic and low linoleic acid corn plants have been reported in U.S. Pat. No. 6,248,939. Disclosed therein is a corn plant capable of producing grain having a ten-fold increase in oleic acid content over normal corn by breeding a high oil corn variety with a corn variety that carries a chemically mutated gene that confers high oleic acid content. Specifically, the resulting grain contains about 60% of the total oil consisting of oleic acid. Plants of this type can be used to pollinate high yielding, commercially acceptable hybrids that are male sterile, which have high oleic acid producing characteristics, thus producing grain having a five-fold increase in oleic acid content over that of standard yellow corn.

Plants of the present invention are produced by selective breeding and genetic engineering methods known to those of skill in the respective arts. In the present invention genetically engineered plants are generated using isolated nucleic acid molecules, in sense or antisense orientation. The nucleic acid molecules in the present invention include, but are not limited to, nucleic acids encoding polypeptides affecting the levels of oil, linoleic acid, stearic acid, and xanthophylls in a corn kernel. Exemplary nucleic acid molecules include but are not limited to phytoene synthase, oleoyl-ACP thioesterase, palmitoyl-ACP thioesterase, and Δ⁹-desaturase. The nucleic acid molecules of the present invention also include fragments of nucleic acid molecules encoding polypeptides affecting the levels of oil, linoleic acid, stearic acid, and xanthophylls in a corn kernel. Said fragments of nucleic acid molecules may be used in antisense or other suppression strategies.

As used herein, a phenotypic characteristic is “undesirable” if the characteristic causes an increase in the cost of production or a decrease in the value of the product.

Plant Transformation

The nucleic acids of the present invention are cloned into expression vectors and cassettes prior to transformation into plants. The vectors of the present invention include nucleic acids and appropriate regulatory elements operably linked thereto that facilitate efficient expression of the inventive nucleic acids in a corn plant. Vectors useful in the context of the present invention can include such regulatory elements.

Techniques for transforming a plant cell, a plant tissue, a plant organ, or a plant with a nucleic acid construct, such as a vector are known in the art. Such methods involve plant tissue culture techniques, for example. Herein, “transforming” refers to the introduction of nucleic acid into a recipient host and the expression therein.

The plant cell, plant tissue, plant organ, or plant can be contacted with the vector by any suitable means as known in the art. Preferably, a transgenic plant expressing the desired protein is to be produced. Alternatively, a transgenic plant suppressing the desired protein is to be produced. Various methods for the introduction of a desired polynucleotide sequence encoding the desired protein into plant cells include, but are not limited to: (1) physical methods such as microinjection (Capecchi, Cell, 22(2): 479-488 (1980)), electroporation (Fromm et al., Proc. Nat. Acad. Sci. (U.S.A.), 82(17): 5824-5828 (1985); U.S. Pat. No. 5,384,253), and microprojectile mediated delivery (biolistics or gene gun technology) (Christou et al., Bio/Technology, 9:957 (1991); Fynan et al., Proc. Nat. Acad. Sci. (U.S.A.) 90(24): 11478-11482 (1993)); (2) virus mediated delivery methods (Clapp, Clin. Perinatol., 20(1): 155-168 (1993); Lu et al., J. Exp. Med., 178(6): 2089-2096 (1993); Eglitis and Anderson, Biotechniques, 6(7): 608-614 (1988)); and (3) Agrobacterium-mediated transformation methods.

The most commonly used methods for transformation of plant cells are the Agrobacterium-mediated DNA transfer process (Fraley et al., Proc. Nat. Acad. Sci. (U.S.A.), 80:4803 (1983)) and the microprojectile bombardment mediated process. Typically, nuclear transformation is desired but where it is desirable to specifically transform plastids, such as chloroplasts or amyloplasts, plant plastids may be transformed utilizing a microprojectile mediated delivery of the desired polynucleotide for certain plant species.

Agrobacterium-mediated transformation is achieved through the use of a genetically engineered soil bacterium belonging to the genus Agrobacterium. Several Agrobacterium species mediate the transfer of a specific DNA known as “T-DNA,” which can be genetically engineered to carry any desired piece of DNA into many plant species. The major events marking the process of T-DNA mediated pathogenesis are: induction of virulence genes, processing and transfer of T-DNA. This process is the subject of many reviews (Ream, Ann. Rev. Phytopathol., 27: 583-618 (1989); Howard and Citovsky, Bioassays, 12: 103-108 (1990); Kado, Crit. Rev. Plant Sci., 10: 1-32 (1991); Zambryski, Annual Rev. Plant Physiol. Plant Mol. Biol., 43: 465-490 (1992); Gelvin, In Transgenic Plants, Kung and Wu, eds., Academic Press, San Diego, Calif., pp. 49-87 (1993); Binns and Howitz, In Bacterial Pathogenesis of Plants and Animals, Dang, ed., Berlin: Springer Verlag, pp. 119-138 (1994); Hooykaas and Beijersbergen, Ann. Rev. Phytopathol., 32: 157-179 (1994); Lessl and Lanka, Cell, 77: 321-324 (1994); Zupan and Zambryski, Annual Rev. Phytopathol., 27: 583-618 (1995)).

Agrobacterium-mediated genetic transformation of plants involves several steps. The first step, in which the virulent Agrobacterium and plant cells are first brought into contact with each other, is generally called “inoculation.” The Agrobacterium containing solution is then removed from contact with the explant by draining or aspiration. Following the inoculation, the Agrobacterium and plant cells/tissues are permitted to be grown together for a period of several hours to several days or more under conditions suitable for growth and T-DNA transfer. This step is termed “co-culture.” Following co-culture and T-DNA delivery, the plant cells are treated with bactericidal or bacteriostatic agents to kill the Agrobacterium remaining in contact with the explant and/or in the vessel containing the explant If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is typically followed by one or more “selection” steps. Both the “delay” and “selection” steps typically include bactericidal or bacteriostatic agents to kill any remaining Agrobacterium cells because the growth of Agrobacterium cells is undesirable after the infection (inoculation and co-culture) process.

A number of wild-type and disarmed strains of Agrobacterium tumefaciens and Agrobacterium rhizogenes harboring Ti or Ri plasmids can be used for gene transfer into plants. The Agrobacterium hosts contain disarmed Ti and Ri plasmids that do not contain the oncogenes that cause tumorigenesis or rhizogenesis, respectively, which are used as the vectors and contain the genes of interest that are subsequently introduced into plants. Preferred strains would include but are not limited to Agrobacterium tumefaciens strain C58, a nopaline-type strain that is used to mediate the transfer of DNA into a plant cell, octopine-type strains such as LBA4404 or succinamopine-type strains, e.g., EHA101 or EHA105. The nucleic acid molecule, prepared as a DNA composition in vitro, is introduced into a suitable host such as E. coli and mated into the Agrobacterium, or directly transformed into competent Agrobacterium. These techniques are well-known to those of skill in the art.

The Agrobacterium can be prepared either by inoculating a liquid such as Luria Burtani (LB) media directly from a glycerol stock or streaking the Agrobacterium onto a solidified media from a glycerol stock, allowing the bacteria to grow under the appropriate selective conditions, generally from about 26° C. to about 30° C., or preferably about 28° C., and taking a single colony or a small loop of Agrobacterium from the plate and inoculating a liquid culture medium containing the selective agents. Those of skill in the art are familiar with procedures for growth and suitable culture conditions for Agrobacterium as well as subsequent inoculation procedures. The density of the Agrobacterium culture used for inoculation and the ratio of Agrobacterium cells to explant can vary from one system to the next, and therefore optimization of these parameters for any transformation method is expected.

With respect to microprojectile bombardment (U.S. Pat. Nos. 5,550,318; 5,538,880; 5,610,042; and PCT Application WO 95/06128; each of which is specifically incorporated herein by reference in its entirety), particles are coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the microprojectile stopping plate.

In addition to direct transformation of a particular plant genotype with a construct prepared according to the present invention, transgenic plants may be made by crossing a plant having a construct of the present invention to a second plant lacking the construct. For example, a selected coding region operably linked to a promoter can be introduced into a particular plant by crossing, without the need for ever directly transforming the target plant. Therefore, the present invention not only encompasses a plant directly regenerated from cells which have been transformed in accordance with the present invention, but also the progeny of such plants. As used herein the term “progeny” denotes the offspring of any generation of a parent plant prepared in accordance with the present invention, wherein the progeny comprises a construct prepared in accordance with the present invention. “Crossing” a plant to provide a plant line having one or more added transgenes relative to a starting plant line, as disclosed herein, is defined as the techniques that result in one or more transgenes of the present invention being introduced into a plant line by cross pollinating a starting line with a donor plant line that comprises a transgene of the invention. To achieve this one could, for example, perform the following steps:
a) plant seeds of the first (starting line) and second (donor line comprising a transgene of the present invention) parent plants;
b) grow the seeds of the first and second parent plants into plants that bear flowers;
c) pollinate the first parent plant with pollen from the second parent plant; and
d) harvest seeds produced on the first parent plant.

“Backcrossing” is herein defined as the process including the steps of:
a) crossing a plant of a first genotype containing a desired gene, DNA sequence or element to a plant of a second genotype lacking the desired gene, DNA sequence or element;
b) selecting one or more progeny plant containing the desired gene, DNA sequence or element;
c) crossing the progeny plant to a plant of the second genotype; and
d) repeating steps (b) and (c) for the purpose of transferring the desired gene, DNA sequence, or element from a plant of a first genotype to a plant of a second genotype.

Introgression of a DNA element into a plant genotype is defined as the result of the process of the backcross conversion. A plant genotype into which a DNA sequence has been introgressed may be referred to as a backcross converted genotype, line, inbred or hybrid.

Characterization of Transformed Plants

To confirm the presence of the transgene in the regenerated plant, a variety of techniques are available, which are well known in the art. Examples of these techniques include but are not limited to (a) molecular assays of DNA integration or RNA expression such as Southern or northern blotting, TAQMAN® technology (Applied Biosystems, Foster City, Calif.) and PCR; (b) biochemical assays detecting the presence of the protein product such as ELISA, western blotting, or by enzymatic function; or (c) chemical analysis of the targeted plant part, such as seed tissue, for qualitative and quantitative determination of oil, protein, or xanthophylls.

The following examples are provided for illustration purposes, and are not intended to limit the present invention in any way.

### EXAMPLE 1

This example sets forth one method of segmenting a market by producing a specialty feed corn having high oil and low pigmentation, for use in swine production.

The production of corn grain of high oil and low pigmentation can be generally described by the steps: a) development of at least one high oil, low pigment inbred; b) producing hybrid seed by intermating of a high oil, low pigment inbred with an unrelated heterotic inbred; and c) growing of the resulting hybrid seed and harvesting the resulting grain crop.

A high oil, low pigment inbred can be developed by first selecting at least two parental lines with complimentary characteristics for intermating. Among these lines must be included the attributes of high oil and low pigment. An example would be the intermating of the high oil, low pigment source line IHO (available from the National Seed Storage Laboratory as accession 20626) with LH185 (a high yielding modern inbred available commercially from Corn States Hybrid Service, Des Moines, Iowa). Another example would be the intermating of a low xanthophyll content corn line with a high oil source line. Low xanthophylls content corn lines have been described by Strissel (PCT Application WO 02/76188), which is incorporated herein by reference. Exemplary low pigment inbreds used for crossing to high oil inbreds are WICY226C, WEB428C, and WICY418C from Wilson Genetics (Harland, Iowa). Exemplary high oil inbreds are HO1001 and H01002 as described by Foley in U.S. Patent Applications 20030182697 and 20030154524, respectively.

After selecting and intermating the two parental lines with complementary characteristics, the resulting F1 seed is collected. Using the F1 seed, inbred lines with stable genetic complement are generated, most commonly through several generations of self-pollination, but alternatively through successive backcrossing to one of the original parental lines or through dihaploidy. During the multiple generations of inbreeding, progeny are selected for kernels containing the combined attributes of an oil level above about 5% and a pigmentation potential less than that of standard yellow corn.

Hybrid seed having high oil and low pigment kernels are produced by planting, in pollinating proximity, seeds of a first low pigment inbred, and a second corn plant heterotic to it, which is also preferably low in pigmentation. The first and second parent corn plants are then grown into plants that bear flowers. Pollen production is prevented by emasculating flowers of either the first or second parent corn plant. Natural cross-pollination between the first and second parent corn plants is thus allowed to occur, and the seeds of the emasculated parent corn plant are harvested at maturity. Finally, the resulting high oil, low pigment hybrid seeds are cultivated to maturity under common corn grain crop production practices, and the resulting high oil, low pigment grain is harvested.

The high oil, low pigment corn is desirable to the swine producers because of the high metabolizable energy value and the absence of a pigment. The result is an economical source of energy and the resultant meat having a desirable white fat. In contrast, this grain would not be desirable in certain segments of the broiler or egg layer industry because of the absence of the pigment. Although the poultry producer would benefit from the high oil, he would have to add a supplemental source of pigment to the feed, thus adding cost to the feed formulation and making it less desirable.

A high oil, low pigment (LP) corn produced as described above is formulated into a swine feed to replace commodity yellow #2 corn. The following formulation shown in Table 1 delivers an increased energy content, relative to typical yellow #2 corn while providing a minimal amount of fat-soluble pigment.

TABLE 1
Feed components

| | Yellow #2 corn | High oil LP corn |
|---|---|---|
| High oil LP corn | — | 1,456 |
| Yellow #2 corn | 1,456 | — |
| 47% soybean meal | 440 | 440 |
| Meat and Bone meal | 40 | 40 |
| Lysine HCl, 78.5% | 4 | 4 |
| Swine Vitamin mineral premix | 60 | 60 |
| Total, lbs | 2,000 | 2,000 |

Calculated dietary nutrient analysis of feed

| | Yellow #2 corn | High oil LPP corn |
|---|---|---|
| Metabolizable energy, Kcal/kg | 3,285 | 3,423 |
| Crude Protein, % | 17.56 | 17.92 |
| Calcium, % | 0.70 | 0.70 |
| Phosphorus, % | 0.53 | 0.53 |
| Lysine, % | 0.94 | 0.94 |
| Xanthophyll content, mg/kg | 13.10 | 3.65 |

Nutrient analysis of corn source used in previous example

| | Yellow #2 corn | High oil white corn |
|---|---|---|
| Metabolizable energy, Kcal/kg | 3,420 | 3,570 |
| Moisture, % | 12.00 | 12.00 |
| Crude Protein, % | 8.30 | 8.80 |
| Oil, % | 3.50 | 6.50 |
| Calcium, % | 0.03 | 0.03 |
| Phosphorus, % | 0.28 | 0.28 |
| Lysine, % | 0.26 | 0.26 |
| Xanthophyll content, mg/kg | 18.00 | 5.00 |

Notes:
Typical 50-100 lb. growing pig ration.
LP corn has a pigmentation potential less than that of standard yellow corn.

### EXAMPLE 2

This example sets forth one method of segmenting a market by producing a corn having a high pigment content, for use in a poultry feed ration.

High carotenoid corn plants are described by Shewmaker in PCT Application WO 01/88169, which is incorporated herein by reference. For expression of phytoene synthase in the corn endosperm, the crtB coding sequence from E. herbicola (PCT Application WO 91/13 078, Armstrong et al. (1990)) is cloned such that it is expressed under control of the rice glutelin (pGtl) promoter (Leisy et al., Plant Mol. Biol., 14: 41-50 (1989)), and the HSP70 intron sequence U.S. Pat. No. 5,593,874, which are herein incorporated by reference in their entirety. This cassette also includes the transcriptional termination region downstream of the cloning site of nopaline synthase, nos Y, (Depicker et al., J. Molec. Appl. Genet., 1: 562-573 (1982)), to create the vector for transformation into corn.

Transgenic corn plants are produced by an Agrobacterium-mediated transformation method. A disarmed Agrobacterium strain C58 (ABI) harboring a binary vector is used. The prepared corn transformation vector is transferred into Agrobacterium by a triparental mating method (Ditta et al., Proc. Natl. Acad. Sci. (U.S.A.), 77: 7347-7351 (1974)), which is incorporated herein by reference.

Prior to inoculation of maize cells the Agrobacterium cells are grown overnight at room temperature in AB medium (Chilton et al., Proc. Natl. Acad. Sci. (U.S.A.), 71: 3672-3676 (1974)), comprising appropriate antibiotics for plasmid maintenance and 200 μM acetosyringone. Immediately prior to inoculation the Agrobacterium cells are pelleted by centrifugation and resuspended in ½ MSVI medium (2.2 g/L MS basal salts (Murashige and Skoog, Physiol. Plant, 15: 473-497 (1962)), 2 mg/L glycine, 0.5 g/L niacin, 0.5 g/L L-pyridoxin-HCl, 0.1 mg/L thiamine, 115 g/L L-proline, 10 g/L D-glucose, and 10 g/L sucrose, pH 5.4) containing 200 μM acetosyringone.

The immature maize embryos are excised and incubated in the Agrobacterium suspension, described above, at room temperature for approximately 5 minutes.

Following Agrobacterium infection and co-culture, the embryos are transferred to type II delay medium and cultured at 27° C. in the dark for 5 to 7 days. The delay medium consists of MS basal salts containing 2.0 mg/L 2,4-D (GIBCO), 100 mg/L-casamino acids, 12 mM proline, 500 mg/L carbenicillin and 20 μM silver thiosulfate. Once signs of type II callus initiation from immature embryos are observed, as defined by Selman et al., in The Maize Handbook, Freeling and Walbot, eds., Springer Verlag, p. 672 (1994), the coleoptiles are removed from the embryos. The embryos are then transferred to MS medium containing 2.0 mg/L 2,4-D, 12 mM proline, 20 μM silver thiosulfate, 500 mg/L carbenicillin and 0.5 mM glyphosate (Monsanto Company, St. Louis, Mo.) and incubated at 27° C. in the dark for 2 weeks.

Embryos forming callus are transferred to the MS medium described above, but additionally containing 1.0 mM glyphosate. The cultures are then incubated for 2 weeks in the dark at 27° C. The embryos still having callus are then transferred to MS medium containing 3.0 mM glyphosate for an additional 2 weeks.

Seeds from the resulting transgenic corn lines are analyzed for carotenoid levels. Total carotenoid concentrations are increased up to 5-fold over control plants. Up to a 15-fold increase is observed with phytoene levels.

Corn grain having a pigmentation potential greater than that of standard yellow corn, produced as described above, can be sold as a grain meal or a feed source to poultry producers. The high carotenoid level is an added value to the poultry producer because it serves as a pigmentation source. The pigmentation enhances the color of the final product, making it more desirable to the consumer. On the other hand the high pigment content corn would not be desirable to the pork producer because of the undesirable pigmentation of the fat layer.

The high pigment content corn, as described above, can also be crossed into or produced in a high oil corn germplasm. The resulting phenotype of high oil and high pigment content would be another example of grain manipulated to be more adapted to a first market segment and less adapted to a second market segment. In this example, the high pigment content corn, with or without the high oil phenotype, is more adapted to the poultry market and less adapted to the swine market.

### EXAMPLE 3

This example describes a method of producing a corn with altered linoleic acid levels, specifically for use in swine feed rations.

Corn is modified to have a reduced linoleic acid content by the method set forth by Rubin-Wilson (U.S. Pat. No. 6,331,664). This strategy uses genes encoding maize oleoyl-ACP and palmitoyl-ACP thioesterase enzymes isolated from maize, that when expressed in a plant, can be used to create transgenic plants having reduced linoleic acid oil profiles.

Linoleic acid is an essential fatty acid that is necessary for normal growth, development, and reproduction. However, the amount of dietary linoleic acid required varies by species. For example, the National Research Council (Poultry NRC, 1994) recommends that the diet for growing broilers contain a minimum of 1% linoleic acid (w/w). By contrast, the NRC (Swine NRC, 1998) has a minimum dietary recommendation for linoleic acid in swine rations of 0.1% (w/w). Linoleic acid levels could, therefore, be used as a trait and a means to segment markets for specialty corn. An example would be to produce a corn having normal oil levels (typically 3.5% w/w) and low linoleic acid levels (less than 10% of the total fatty acid). The corn would be desirable to swine producers that would realize the dietary requirement for 18:2 fatty acid, and may also have a beneficial effect on the fat quality by lowering the iodine value of the carcass. The poultry producers would not find it desirable to feed this grain because the linoleic acid levels would be too low to meet NRC requirement.

The low linoleic acid corn described above is crossed into a high oil germplasm, and the resultant grain is formulated into a swine feed to replace commodity yellow #2 corn. The following formulation in Table 2 delivers a similar energy content without providing a detrimental level of linoleic acid (18:2).

TABLE 2

| | |
|---|---|
| | High oil, |
| Yellow #2 corn | low linoleic |

Feed Components

| | | |
|---|---|---|
| High oil, low 18:2 | — | 1,456 |
| Yellow #2 corn | 1376 | — |
| 47% soybean meal | 460 | 440 |
| Meat and Bone meal | 40 | 40 |
| Animal-vegetable grease | 60 | — |
| Lysine-HCl, 78.5% | 4 | 4 |
| Swine Vitamin mineral premix | 60 | 60 |
| Total, lbs | 2,000 | 2,000 |

Calculated dietary nutrient analysis of feed

| | | |
|---|---|---|
| Metabolizable energy, Kcal/kg | 3,429 | 3,423 |
| Crude Protein, % | 17.70 | 17.92 |
| Calcium, % | 0.70 | 0.70 |
| Phosphorus, % | 0.53 | 0.53 |
| Lysine, % | 0.94 | 0.94 |
| Linoleic acid, % | 2.48 | 0.34 |

Nutrient Analysis of corn source used in previous example

| | | |
|---|---|---|
| Metabolizable energy, Kcal/kg | 3,420 | 3,570 |
| Moisture, % | 12.00 | 12.00 |
| Crude Protein, % | 8.30 | 8.80 |
| Oil, % | 3.50 | 6.50 |
| Calcium, % | 0.03 | 0.03 |
| Phosphorus, % | 0.28 | 0.28 |
| Lysine, % | 0.26 | 0.26 |
| Oil fatty acid profile (% of oil) | | |
| Palmitate | 12.50 | 12.00 |
| Stearate | 2.50 | 35.00 |
| Oleic | 29.00 | 46.00 |
| Linoleic acid | 55.00 | 5.00 |

Typical 50-100 lb. growing pig ration.

The resulting feed is sold as a differentiated product into the swine production market. The swine producer realizes an increased energy density in feed rations while maintaining a desired meat product having an improved fat firmness index or iodine value due to dramatically reduced linoleic acid levels in the diet (Pig Improvement Company USA, T&D Technical Memo, Lexington, Ky. (1998)). Premium pork product sold to Japan needs to have iodine values of about 70 or less. Pigs that consume diets containing supplemental energy sources such as vegetable oils, poultry grease, lard, and restaurant grease have difficulties meeting this requirement due to the elevated levels of linoleic acid, which the animal deposits in its subcutaneous fat stores, thereby negatively affecting the fat firmness index.

Linoleic acid levels of commonly used supplemental fat sources in livestock diets (Poultry NRC, 1994) are shown in Table 3.

TABLE 3

| | Linoleic acid |
|---|---|
| | content, % |
| Tallow | 3.0 |
| Lard | 10.5 |
| Poultry grease | 23.5 |
| Animal/Vegetable | 29.0 |
| grease blend | |
| Soybean oil | 53.0 |

A poultry producer would be less interested in feeding this reduced linoleic acid corn in a broiler or layer ration because of the need to supplement linoleic acid necessary to meet the higher nutritional requirement of poultry as compared to swine. The need for supplemental linoleic acid would make the corn uneconomical compared to other higher energy (fat) sources, which contain adequate levels of linoleic acid.

### EXAMPLE 4

This example describes two methods of producing corn having high stearate levels for use in swine feed rations.

Corn with increased levels of stearate has been demonstrated by Zwick et al., U.S. Patent Application 20030014775, and by Shen, PCT Application WO 99/64579. Zwick and Shen, which are herein incorporated by reference, each disclose increased stearate levels by suppressing the Δ⁹-desaturase activity in corn.

In a first example, Zwick discloses the use of ribozymes to inhibit Δ⁹-desaturase activity. Ribozymes are endogenously expressed RNA molecules containing substrate binding domains that bind to accessible regions of the target mRNA. The ribozymes also contain domains that catalyze the cleavage of RNA. The described ribozymes are preferably ribozymes of the hammerhead (HH) or hairpin (HP) motif. Upon binding, the ribozymes cleave the target mRNAs, preventing translation and protein accumulation. Thus, using ribozymes, the Δ⁹-desaturase activity was reduced or eliminated resulting in increased stearate levels and inhibited unsaturated fatty acid production.

Zwick used an isolated Δ⁹-desaturase cDNA from corn plants to identify HH and HP ribozyme sites in corn Δ⁹-desaturase mRNA. The Δ⁹-desaturase cDNA from corn plants was isolated using standard molecular biology techniques (for example, see Sambrook, J., Fritsch, E. F., and Maniatis, T. (1989), Molecular Cloning a Laboratory Manual, second edition, Cold Spring Harbor: Cold Spring Harbor Laboratory Press, which is incorporated herein by reference). Briefly, using degenerate PCR primers designed and synthesized to two conserved peptides involved in diiron-oxo group binding of plant Δ⁹-desaturase, a 276 bp DNA fragment was PCR amplified from maize embryo cDNA and cloned into a vector. This sequence was used to screen a maize embryo cDNA library. A clone was isolated and sequenced, and the sequence of this clone was used to identify HH and HP ribozyme sites in corn Δ⁹-desaturase mRNA. The secondary structure of Δ⁹-desaturase mRNA was assessed by computer analysis using algorithms, such as those developed by Zuker, Science, 244: 48-52 (1989).

Both HH and HP ribozymes were designed and chemically synthesized. The general procedures for RNA synthesis have been described previously, (Usman et al., J. Am. Chem. Soc., 109: 7845-7854, (1987) and in Scaringe et al., Nucl. Acids Res., 18: 5433-5341, (1990); Wincott et al., Nucleic Acids Res., 23:2677, (1995)).

Using these ribozymes, corn callus was transformed and regenerated into plantlets. Six ribozyme constructs targeted to Δ⁹-desaturase were transformed into embryogenic, regenerable type II callus cultures. Independent transformation events were isolated from the selection, and lines were chosen for regeneration, and R0 plants are produced.

From the R0 plants, leaf tissue was assayed for stearic acid as described by Browse et al., Anal. Biochem., 152: 141-145 (1986). Plants were assayed from lines transformed with active Δ⁹-desaturase ribozymes and from lines transformed with Δ⁹-desaturase inactive ribozymes. When compared to the control and inactive Δ⁹-desaturase ribozyme data, the results for the stearic acid content in the active lines demonstrated an increase in stearic acid content due to the introduction of the Δ⁹-desaturase ribozyme.

As a second example, Shen discloses corn callus transformed with a vector containing a Δ⁹-desaturase gene in antisense orientation, using a particle bombardment method, (BioRad Instruments, Hercules, Calif.). Following transformation, stable transformants were selected and transgenic corn plants regenerated. Primary corn plants (R0 plants) were grown in the greenhouse. The plants were then either selfed or crossed using wild type pollen (Holdens inbred line LH132, Monsanto Company, St. Louis, Mo.). The cobs from these plants were harvested at 30 days after planting (DAP). The embryos were dissected out of the kernels and sterilized. Small pieces of scutella were taken from each individual embryo and used for fatty acid composition assays by the GC method set forth in PCT Application WO 94/11516. The embryos with a high level of saturated fatty acids in the lipid fraction were grown to R1 plants. The mature R1 plants were then selfed. The cobs were harvested at 45 DAP, and fatty acid analysis is performed as described above.

The resulting fatty acid analysis showed that plants having a heterozygous kernel, (the plants crossed with LH132), had a stearate content of 26.1% as compared to a wild type control of 2%. The fatty acid composition of the R2 seed segregants shows that the average stearate composition of the kernels is 37%, as compared to 2% for the controls. These results demonstrate another example of a corn plant with high stearate levels.

### EXAMPLE 5

This example describes a method of using a high stearate trait to segment the poultry and swine feed corn markets.

High stearate corn, as described in Example 4, is produced and marketed as a high value feed corn, specific to pork production. The corn has an increased value to the pork producer because of the decreased amounts of unsaturated fatty acids that result with the increased stearate concentrations. The negative impact of unsaturated fatty acids on pork carcasses is due to poor sliceability of specific meat cuts that are high in interstitial or intermuscular fat such as the belly area which is used for the manufacture of bacon (Pig Improvement Company USA, T&D Technical Memo, Lexington, Ky. (1998); Madsen A. et al., “Influence of dietary fat on carcass fat quality in pig. A review.” Acta Agric Scand. (sect A) Animal Sci., 42: 220-225 (1992); Redshaw, M. S., “Factors in the diet affecting the quality of pig meat,” Doctoral Thesis, University of Nottingham, Sutton Bonington Campus, (1995)).

Increased stearate concentration in corn improves (lowers) the iodine value of the oil fed to pigs. Increased palmitate or a combination of increased stearate, palmitate, and lower linoleic would have similar effects with regards to improving the iodine value and thus carcass quality of pork. The corn with lower iodine value would have a lower metabolizable energy for poultry. This improved oil corn product would, therefore, have a higher value to swine producers, but a lower or discounted value to poultry producers, thus segmenting the market and maintaining maximum value for two distinct markets.

Four examples of corn oil fatty acid profiles that would have improved iodine values for feeding to pigs, but not beneficial to poultry are shown in Table 4.

TABLE 4

| | Typical | Example | | | |
|---|---|---|---|---|---|
| Fatty Acid, Oil % | Corn Oil | 1 | 2 | 3 | 4 |
| Palmitate | 12.5 | 55.0 | 12.0 | 12.0 | 12.0 |
| Stearate | 2.5 | 2.5 | 35.0 | 52.0 | 45.0 |
| Oleic | 29.0 | 29.0 | 47.0 | 21.0 | 23.0 |
| Linoleic | 55.0 | 12.5 | 5.0 | 14.0 | 19.0 |
| Linolenic | 0.5 | 0.5 | 0.5 | 0.5 | 0.5 |
| Iodine value | 137 | 51 | 53 | 46 | 56 |

The high stearate phenotypic trait can be used as a sole trait or in combination with other traits described in previous examples. For example, a high stearate-low pigment corn would be very desirable to the pork producer but not to the poultry producer. High stearate-low pigment corn therefore would be one example of the present invention wherein a market could be segmented by manipulating two phenotypic characteristics of a grain, wherein the manipulated grain is more adapted to the swine feed market segment and less adapted to the poultry feed market segment as compared to a non-manipulated grain.

1. A method of segmenting a market for a grain comprising manipulating at least one phenotypic characteristic of the grain, wherein the manipulated grain is more adapted to a first market segment and less adapted to a second market segment as compared to a non-manipulated grain, and wherein at least one of said phenotypic characteristics is undesirable to said second market segment.

2. The method of claim 1, wherein the phenotypic characteristic is selected from the group consisting of oil content, pigmentation potential, linoleic acid content, and iodine value.

3. The method of claim 2, wherein the phenotypic characteristic is pigmentation potential.

4. The method of claim 3, wherein the pigmentation potential of said grain is less than that of standard yellow corn.

5. The method of claim 4, wherein the grain has a xanthophyll concentration of less than about 15 ppm.

6. The method of claim 4, further comprising a second phenotypic characteristic consisting of iodine value and wherein the iodine value is less than about 70.

7. The method of claim 4, 5, or 6, wherein the first market segment is swine feed and the second market segment is poultry feed.

8. The method of claim 3, wherein the pigmentation potential is greater than that of standard yellow corn.

9. The method of claim 8, further comprising a second phenotypic characteristic consisting of oil content, and wherein the oil content is greater than about 5%.

10. The method of claim 8 or 9, wherein the first market segment is poultry feed and the second market segment is swine feed.

11. The method of claim 2, wherein the phenotypic characteristic is iodine value.

12. The method of claim 11, wherein the iodine value is less than about 70.

13. The method of claim 12, wherein the grain further comprises a stearate fraction between about 8% and about 80%.

14. The method of claim 12 or 13, wherein the first market segment is swine feed and the second market segment is poultry feed.

15. The method of claim 12, further comprising a second phenotypic characteristic consisting of pigmentation potential, and wherein the pigmentation potential is less than that of standard yellow corn.

16. The method of claim 15, wherein the grain further comprises a pigment content having a xanthophyll concentration of less than about 15 ppm.

17. The method of claim 15 or 16, wherein the first market segment is swine feed and the second market segment is poultry feed.

18. The method of claim 2, wherein the phenotypic characteristic is linoleic acid content.

19. The method of claim 18, wherein the linoleic acid content is decreased to between about 1% and about 30%.

20. The method of claim 19, further comprising a second phenotypic trait consisting of pigmentation potential and wherein the pigmentation potential is less than that of standard yellow corn.

21. The method of claim 19 or 20, wherein the first market segment is swine feed and the second market segment is poultry feed.
